(19) United States
(12) Patent Application Publication
(10) Pub. No.: US 2002/0099402 A1
(43) Pub. Date: Jul. 25, 2002

(54) Methods and apparatus for intercostal access

(75) Inventors: Robert F. Buckman, Radnor, PA (US); Rodney A. Brenneman, San Juan Capistrano, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: THERACARDIA, INC., San Clemente, CA

(21) Appl. No.: 10/025,517

(22) Filed: Dec. 18, 2001

Related U.S. Application Data
(63) Continuation-in-part of application No. 09/768,041, filed on Jan. 22, 2001.

(51) Int. Cl. A61B 17/34

(52) U.S. Cl. 606/185

(57) ABSTRACT

The present invention provides improved devices and methods for establishing intercostal access for subsequent placement of minimally invasive direct cardiac massagers, chest tubes, defibrillation electrodes, and the like. In particular, the present invention provides devices and methods which facilitate rapid, safe, and sterile intercostal dissection for the subsequent deployment of minimally invasive direct cardiac massagers. An intercostal device according to the present invention comprises a support having a proximal end and a distal end, a cutting tip coupled to the distal end of the support, and rib engaging means coupled to the support or cutting tip. The cutting tip is adapted to penetrate percutaneously through intercostal tissue between adjacent ribs to a thoracic cavity over a heart. Means coupled to the support or cutting tip penetrate or dilate tissue, engage at least one rib, and stop advancement of the cutting tip into the thoracic cavity.

### CROSS-REFERENCES TO RELATED APPLICATIONS

This application is a continuation-in-part and claims the benefit of priority from co-pending U.S. patent application Ser. No. 09/768,041, filed Jan. 22, 2001, which is incorporated herein by reference.

### BACKGROUND OF THE INVENTION

1. Field of the Invention

The present invention relates generally to medical devices and methods. More particularly, the present invention relates to improved devices and methods for establishing intercostal access for subsequent placement of minimally invasive direct cardiac massagers, chest tubes, defibrillation electrodes, and the like.

Sudden cardiac arrest is a leading cause of death in most industrial societies. In order to resuscitate a victim of cardiac arrest, it is necessary to provide an adequate artificial circulation of blood to oxygenate the heart and brain by re-establishing the pumping function of the heart during the period between cardiac arrest and restoration of normal cardiac activity. Such a cardiac pumping function must be instituted at the earliest possible state. While in many cases it is theoretically possible to re-establish cardiac function, irreversible damage to vital organs, particularly the brain and the heart itself, will usually occur if sufficient blood flow is not re-established within a critical period of time from the moment of cardiac arrest. Such a period of time is measured ranging between four and six minutes.

A number of techniques have been developed to provide artificial circulation of blood to oxygenate the heart and brain during a cardiac arrest. Of particular interest to the present invention is the recent introduction of devices for performing minimally invasive direct cardiac massage. Such devices and methods are described in co-pending applications Nos. 09/087,665 filed May 29, 1998; 60/111,934 filed Dec. 11, 1998 (now abandoned); 09/344,440 filed Jun. 25, 1999; and 09/356,064 filed Jul. 19, 1999, assigned to the assignee of the present application. Generally, such methods rely on advancing a plurality of struts through an intercostal space to a region over a pericardium. The struts are opened along arcuate radially diverging paths between a posterior rib surface and the pericardium. The heart may then be pumped by directly engaging the opened struts against the pericardium to periodically compress the heart. Alternative minimally invasive direct cardiac massage devices and methods are also described in U.S. Pat. Nos. 5,582,580; 5,571,074; and 5,484,391 issued to Buckman, Jr. et al. and 5,931,850; 5,683,364; and 5,466,221 issued to Zadini et al., licensed to the assignee of the present application.

While direct cardiac massage approaches offer great promise, one issue to be resolved for the success and practical utility of direct cardiac massage devices is establishing safe, rapid, and sterile first entry into the chest cavity for such massagers. Previously proposed methods for gaining access into the chest cavity include sharp dissection with sharp surgical instruments or with a trocar-cannula assembly. However, such sharp dissection methods are disadvantageous as they often depend on blind advancement of a sharp instrument, which carries a risk of puncturing and/or lacerating the heart, coronary vessels, or the surrounding structures. Other proposed methods employ a combination of sharp and blunt dissection to establish intercostal access. These methods typically employ a sharp surgical instrument, such as a surgical knife, lancet, scalpel, blade, and the like, to make a partial incision through the skin overlying the intercostal space, and then advancing a blunt member through the intercostal space above the pericardium. Likewise, these methods also suffer drawbacks as such multi-step procedures are often time-consuming, slow, and conflict with the need for a rapid institution of cardiopulmonary resuscitation. Furthermore, such multi-step approaches still require a certain level of surgical skill to avoid any potential injuries to intrathoracic organs. Such methods in general also require the use of surgical gloves, which in turn adds to the set up time for a resuscitation procedure where the need for performing urgently is critical. Moreover, these access protocols are not inherently antiseptic, which is an important safeguard as most cardiac arrest occur outside of a hospital setting. Thus, none of the prior art methods or devices have been entirely satisfactory.

For these reasons, it would be desirable to provide improved devices and methods for establishing intercostal access for subsequent placement of minimally invasive direct cardiac massagers, chest tubes, defibrillation electrodes, and the like. In particular, it would be desirable to provide an apparatus and methods which facilitate rapid, safe, and sterile first entry into the chest cavity for the subsequent deployment of minimally invasive direct cardiac massagers. It would be further desirable if such devices minimize the level of surgical skill required for implementation and eliminate the need for surgical gloves. The devices and methods should allow for sharp swift access without injury to intrathoracic organs while maintaining an antiseptic environment. At least some of these objectives will be met by the devices and methods of the present invention described hereinafter.

2. Description of the Background Art

Devices and methods for minimally invasive direct cardiac massage through intercostal dissection are described co-pending applications Nos. 09/087,665 filed May 29, 1998; 60/111,934 filed Dec. 11, 1998 (now abandoned); 09/344,440 filed Jun. 25, 1999; and 09/356,064 filed Jul. 19, 1999. Devices and methods for minimally invasive direct cardiac massage through blunt first entry methods are described by Zadini et al. in U.S. Pat. Nos. 5,931,850; 5,683,364; and 5,466,221, licensed to the assignee of the present application. Devices and methods for minimally invasive direct cardiac massage through sharp intercostal dissection methods are described by Buckman, Jr. et al. in U.S. Pat. Nos. 5,582,580; 5,571,074; and 5,484,391. U.S. Pat. No. 3,496,932, issued to Prisk describes a trocar-cannula assembly for introducing a cardiac massage device to a space between the sternum and the heart. Dissectors employing inflatable components are described in U.S. Pat. Nos. 5,730,756; 5,730,748; 5,716,325; 5,707,390; 5,702,417; 5,702,416; 5,694,951; 5,690,668; 5,685,826; 5,667,520; 5,667,479; 5,653,726; 5,624,381; 5,618,287; 5,607,443; 5,601,590; 5,601,589; 5,601,581; 5,593,418; 5,573,517; 5,540,711; 5,514,153; and 5,496,345.

The full disclosures of each of the above references are incorporated herein by reference.

### SUMMARY OF THE INVENTION

The present invention provides improved devices and methods for establishing intercostal access for subsequent placement of minimally invasive direct cardiac massagers, chest tubes, defibrillation electrodes, and the like. In particular, the present invention provides devices and methods which facilitate rapid, safe, and sterile intercostal dissection for the subsequent deployment of minimally invasive direct cardiac massagers. Moreover, the present invention minimizes the level of surgical skill required for implementation of the present device and eliminates the need for surgical gloves.

In a first aspect of the present invention, an intercostal access device comprises a handle having a proximal end and a distal end, a cutting tip coupled to the distal end of the handle, and rib engaging means coupled to the handle or cutting tip. The cutting tip, which may comprise a blade, trocar, or scalpel, is adapted to penetrate percutaneously through intercostal tissue between adjacent ribs to a thoracic cavity over a heart. Means coupled to the handle or cutting tip engage at least one rib and stop advancement of a leading tip of the cutting tip into the thoracic cavity.

The present access device provides many significant advantages. For example, the access device utilizes the ribs as a reference point so that means coupled to the handle or cutting tip penetrate tissue and engage at least one rib to stop advancement of the cutting tip into the thoracic cavity, regardless of individual variability in the tissue overlying the anterior surface of the rib and/or the depth of the thoracic cavity. As such, rapid sharp dissection with the cutting tip can be safely implemented without fear of blind advancement and/or accidentally puncturing intrathoracic organs. Moreover, a safe and rapid access device improves the effectiveness and usefulness of subsequent direct cardiac massage, where the need for performing urgently and safely is critical. Additionally, implementation of the present access device minimizes the need for specialized surgical skill and, consequently, the device of the present invention may be applied by semi-skilled persons, such as paramedic personnel and the like. This will ultimately effect the rapidity of dissection, and hence, the usefulness of the device.

The rib engaging means to stop advancement of a leading tip of the cutting tip can take a variety of forms. Conveniently, it can be a shoulder formed integrally with the cutting tip in a single blade structure. The blade will penetrate tissue across a width larger than an intercostal space. The shoulder(s) will engage anterior surface(s) of rib(s) before the leading tip of the cutting tip penetrates too far, i.e., before a leading tip has reached the thoracic cavity (typically being within ±5 mm of the cavity, usually within ±3 mm of the cavity). In an exemplary embodiment, the rib engaging means to stop advancement of the cutting tip into the thoracic cavity takes the form of a cylindrical structure which is formed separately from the cutting tip. Such a separate structure mechanically couples the handle to the cutting tip so that the structure will separate or dilate tissue when the cutting tip is advanced and engage an anterior rib surface to halt advancement of the cutting tip at an appropriate point, i.e., when a leading tip of the cutting tip reaches a preselected intercostal depth (typically being in a range from about 2 mm to about 15 mm from an outer rib surface).

The cylindrical structure may be removably attachable to the distal end of the handle. It will be appreciated by those of skill in the art that there are numerous ways to removably couple the structure to the handle, such as, a pressure fit, latch mechanism, cross pin mechanism, and the like. Preferably, the cylindrical structure is removably coupled to the handle by a cross pin configuration. The cylindrical structure has a first hole which is alignable with another hole on the distal end of the handle for receiving a handle pin so that the cylindrical structure is secured within the handle. As such, the present invention advantageously allows for the cylindrical structure and the cutting tip to be separable from the handle after use via removal of the handle pin. This in turn allows the cutting tip and the cylindrical structure to be easily disposed of in a sharps container, as conventional sharps containers may not allow for disposal of the whole access device unit due to size constraints. This removability feature may also allow the handle to be reusable once it has been uncoupled from the cylindrical structure and the cutting tip after use and undergone autoclaving or other sterilization procedures. The handle will usually comprise a shaft or support structure which may have a generally oval shape so as to provide a comfortable and ergonomically correct fit to a user of the device. The handle's oval cross-section has a longitudinal axis which may be alignable with the cutting tip to facilitate orientation of the cutting tip. The handle may additionally have ribs or strips on an outer surface thereof to facilitate gripping of the access device.

The cylindrical structure may further act as a cutting tip holder by having a slot, opening, hole, gap, or slit on a distal end thereof for receiving a proximal end of the cutting tip, wherein the cutting tip is secured within the cylindrical structure by a cutting tip dowel or pin or any other mechanical coupling mechanism. The cylindrical structure has a second and third hole which are alignable with two holes of the cutting tip for receiving the cutting tip dowels or pins. In some instances, the cutting tip may also be independently separable from the cylindrical structure after use via removal of the cutting tip dowels. Hence, the handle, the cylindrical structure, and the cutting tip may be fixed relative to one another during use, wherein the cutting tip remains in an exposed position. Before and after use, device components may be uncoupled from one another so that the cutting tip, the cylindrical structure, and/or the handle may be separately stored or disposed of. In other instances, however, it may be desirable to have the handle, cylindrical structure, and the cutting tip integrally formed as a single unit.

The access device may utilize various cutting tip configurations, typically comprising one to three blades or trocars. In a preferred embodiment the access punch comprises a single blade. The cutting tip will preferably comprise a triangular blade having cutting edges which may be slightly curved or straight and a leading end which includes but in not limited to a pointed tip. The leading point will typically have a maximum length L of 10 mm or less, preferably a maximum length of 6 mm or less, from the distal end of the cylindrical structure or a lateral extension of the cutting tip to further ensure that the cutting tip does not accidentally puncture the heart or lungs. In the exemplary embodiment described above, the cutting tip will be alignable parallel to the ribs, wherein cutting tip orientation may further be facilitated by the oval cross-section handle.

The access device of the present invention may further include a recessed housing structure coupled to the handle for housing the cutting tip before intercostal penetration. Preferably, the housing structure will have a generally bell shape and will be flexible. The recessed housing structure advantageously provides a sterile enclosure for the cutting tip prior to use and acts as a protective cover for the user and during storage. A resilient spring may also be disposed on the handle and rest against the housing structure. The spring will automatically retract the cutting tip back into the housing structure following intercostal penetration.

To promote sterility of the presently claimed access device, a bottom side of the recessed housing structure may be enclosed by having a first penetrable cover or membrane disposed over at least a bottom side of the housing structure, and a second cover disposed over the first cover. The second cover may additionally have a pull-off loop or tab attached to it. It will be appreciated that the covers may be separate components or formed integrally with the housing structure. The first and second covers may optionally be disposed over part of the device or over the entire structure of the device. Suitable materials for the membranes include latex, polyethylene, polypropylene, polyester, and the like. The access device may further comprise an outer pouch or bag which encloses at least a bottom portion of the housing structure. An important advantage of such dual covers and/or an outer pouch is that such structures provide and maintain a sterile environment before and during use of the access device. This is particularly beneficial as most cardiac arrest occur outside a hospital setting in the field. Furthermore, the sterile covers and/or outer pouch eliminate the need for additional packaging and/or surgical gloves, which in turn reduces the set-up time of such procedures and increases the rapidity of device implementation.

The access device may additionally comprise a detachable adhesive skin contacting surface which may be a part of the first cover or the housing structure. The adhesive skin contacting surface will typically form a patch around the dissected tissue to maintain near normal inter-thoracic pressure. As another alternative, the housing structure of the access punch device may have a bottom portion which is detachable from a top portion of the housing structure to form an access port over the dissected tissue. An access port allows for subsequent entry of devices such as minimally invasive direct cardiac massagers, chest tubes, defibrillator electrodes, and the like.

In a second aspect of the present invention, an intercostal access punch comprises a handle having a proximal end and a distal end, a blade holder removably coupled to the distal end of the handle, and a blade coupled to a distal end of the holder. The blade has a leading tip sized to advance through intercostal tissue between adjacent ribs and the blade holder is positioned to engage at least one rib and stop advancement of the leading tip at a preselected intercostal depth as the blade is percutaneously advanced.

In a third aspect of the present invention, an intercostal access punch comprises a handle having a proximal end and a distal end, a blade holder removably coupled to the distal end of the handle, and a blade removably coupled to a distal end of the holder. The blade has a leading tip and at least one shoulder. The leading tip is sized to advance between adjacent ribs and the shoulder is positioned to engage at least one rib and stop advancement of the leading tip prior to entry of the leading tip into a thoracic cavity as the cutting tip is percutaneously advanced toward a heart and internal organs.

In a fourth aspect of the present invention, methods for establishing percutaneous intercostal access are provided. One method comprises advancing a cutting tip having a rib engaging stop element coupled to the cutting tip through intercostal tissue. The stop element engages at least one rib after the cutting tip dissects tissue between adjacent ribs but before the cutting tip enters a thoracic cavity.

The advancing step may be carried out by moving a handle coupled to the cutting tip by a cutting tip holder in a rocking motion so that the cutting tip effectively cuts through the skin, subcutaneous fat, and muscle tissue to a preselected intercostal tissue depth external to the thoracic cavity. The advancing step will typically comprise dilating or separating the tissue with the cutting tip holder as the cutting the tip is percutaneously advanced. Following establishment of the intercostal access tract, the cutting tip may be uncoupled from the handle by removing the cross pin from the handle so that the cutting tip and holder may be disposed of in a conventional sharps container and the handle reused.

Alternatively, the advancing step may be carried out by pressing a housing structure against a skin surface and depressing a moveable handle attached to the cutting tip so that the cutting tip cuts through the skin surface to a preselected tissue depth external to the thoracic cavity. Typically, the amount of force required to depress the handle will be in a range from 0.5 lbs to 3 lbs. Preferably, the advanced cutting tip is automatically retracted back into the housing structure via a resilient spring. The resilient spring will typically require 0.25 lbs to 1 lbs of force to be fully compressed. Preferably, an outer pouch disposed over at least a bottom portion of the housing structure may be removed prior to advancing the cutting tip. An outer cover disposed on a bottom side of the housing structure may also be removed via a pull-off loop or tab prior to cutting tip advancement. The cutting tip may also cut through an inner cover enclosing a bottom side of the housing structure prior to cutting through the skin surface to the preselected tissue depth.

Cutting tip penetration may be limited to a preselected intercostal tissue depth in a range typically from 2 mm to 50 mm, preferably in a range from 2 mm to 15 mm, below an outer rib surface. The preselected tissue depth will usually comprise tissue near an inner rib surface, or may alternatively comprise a thoracic pleural lining. Still further the preselected tissue depth may be within a range from 3 mm to 5 mm from a thoracic cavity. The cutting tip may further be aligned with the ribs prior to cutting tip advancement so the that the cutting tip cuts in a plane parallel to the ribs. Such cutting tip orientation may be desirable as an access incision that lies in a plane parallel to the ribs allows for improved suturing and healing of the thoracic tissue post treatment.

Following cutting tip advancement, an adhesive skin contacting surface of the inner cover or housing structure may be detached on the skin surface so as to form an access patch around the dissected tissue. Optionally, a bottom portion of the housing structure may be detached from a top portion of the housing structure so as to form an access port over the dissected tissue. Still further optionally, a direct cardiac massage device or chest tube may be advanced following intercostal dissection.

### BRIEF DESCRIPTION OF THE DRAWINGS

FIG. 1 is a perspective view of an access punch device for establishing intercostal access constructed in accordance with the principles of the present invention.

FIG. 2 is a bottom view of the device of FIG. 1.

FIG. 3 illustrates an alternate embodiment of the device of the present invention utilizing two blades.

FIG. 4 is a bottom view of the device of FIG. 3.

FIGS. 5A-5C illustrate alternative cutting tip configurations which may be employed in the device of FIGS. 1 or 3.

FIGS. 6A-6B illustrate a detachable adhesive skin contacting surface which may be employed in the device of FIGS. 1 or 3.

FIGS. 7A-7B illustrate a detachable housing structure which may be employed in the device of FIGS. 1 or 3.

FIG. 8 is a cross-sectional view illustrating the heart underneath a patient's rib cage.

FIGS. 9 and 10 illustrate a method according to the present invention employing the access punch device of FIG. 1.

FIGS. 11A and 11B illustrate an exemplary intercostal access device constructed in accordance with the principles of the present invention.

FIGS. 12A and 12B illustrate an alternative configuration of the device wherein the cutting tip holder is removably coupled to the handle by a pin configuration.

FIGS. 13A and 13B illustrate side views of the cutting tip holder of FIG. 11A.

FIGS. 14A and 14B illustrate alternative cutting tip configurations which may be employed in the device of FIG. 11A.

FIG. 14C illustrates a side view of the cutting tip of FIG. 11A.

FIGS. 15A and 15B illustrate methods according to the present invention employing the exemplary access device of FIG. 11A.

### DESCRIPTION OF THE SPECIFIC EMBODIMENTS

The present invention provides improved devices and methods for establishing intercostal access for subsequent placement of minimally invasive direct cardiac massagers, chest tubes, defibrillator electrodes, and the like. In particular, the present invention provides devices and methods which facilitate rapid, safe, and sterile intercostal dissection for the subsequent deployment of minimally invasive direct cardiac massagers.

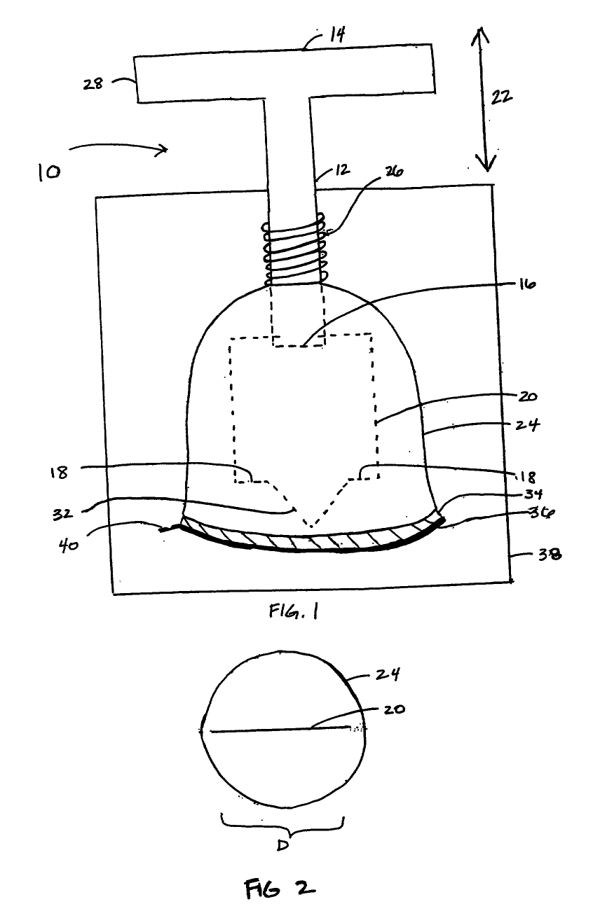

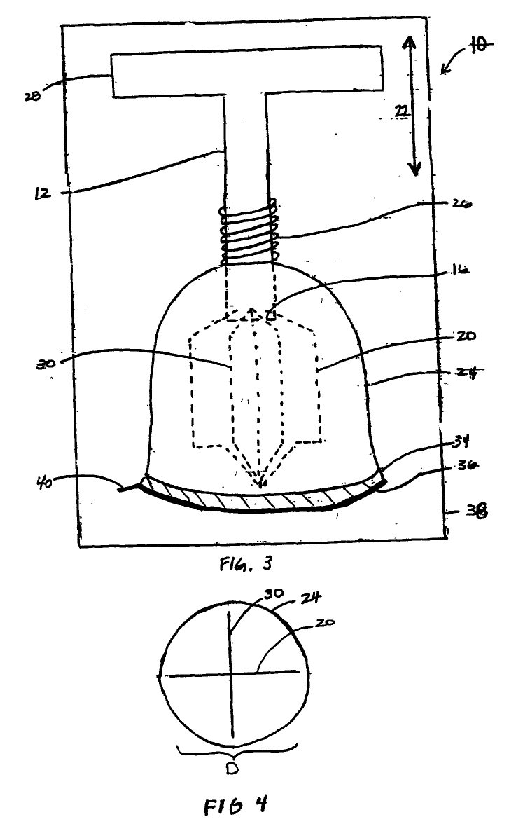

Referring now to FIGS. 1 and 2, an access punch device 10 constructed in accordance with the principles of the present invention for establishing intercostal access is illustrated. The access punch device 10 comprises a support or shaft 12 having a proximal end 14 and a distal end 16, a cutting tip 20 attached to the distal end 16 of the support 12, and means 18 coupled to the cutting tip 20, as shown in FIG. 1, or alternatively to the support 12. The cutting tip 20, which may comprise a blade, scalpel, or trocar, is adapted to penetrate percutaneously through intercostal tissue between adjacent ribs to a thoracic cavity over a heart. Means 18 coupled to the cutting tip 20 or the support 12 penetrate tissue, engage at least one rib, and stop advancement of the cutting tip 20 into the thoracic cavity. As discussed above, the access punch device 10 utilizes the ribs as a reference point so that means 18 coupled to the cutting tip 20 or the support 12 penetrate tissue and engage at least one rib to stop advancement of the cutting tip 20 above the thoracic cavity, regardless of individual variability in the anterior surface of the rib and/or the depth of the thoracic cavity. As such, rapid sharp dissection with the cutting tip 20 and the means 18 coupled to the cutting tip 20 or the support 12 can be safely implemented without fear of blind advancement and/or accidentally puncturing intrathoracic organs. Additionally, implementation of the present access punch device 10 minimizes the need for specialized surgical skill. It will be appreciated that the following depictions are for illustration purposes only and does not necessarily reflect the actual shape, size, or dimensions of the access device 10. This applies to all depictions hereinafter.

The tissue penetrating means 18 to stop advancement of a leading tip 32 of the cutting tip 20 can take a variety of forms. The tissue penetrating means 18 may comprise a lateral extension of the cutting tip 20 which is configured to engage an anterior surface of a rib when a leading tip 32 of the cutting tip 20 reaches the thoracic cavity. Alternatively, the tissue penetrating means 18 can comprise a tissue-penetrating pin, blade, electrosurgical tip, or other tissue penetrating structure which is configured to penetrate tissue when the cutting tip 20 is advanced and to engage an anterior surface of a rib when a leading tip 32 of the cutting tip 20 reaches the thoracic cavity. Such separate structure will be mechanically coupled to the cutting tip 20, however, so that the structure will penetrate tissue and eventually engage an anterior rib surface to halt advancement of the cutting tip 20 at the appropriate point. Optionally, a separate tissue penetrating structure can be positionally adjustable relative to the tissue penetrating tip to account for patients having different body sizes.

The access punch device 10 of the present invention may further include a recessed housing structure 24 attached to the support 12 for housing the cutting tip 20 before intercostal penetration, as illustrated in FIG. 2. Preferably, the housing structure 24 will have a generally bell shape and be made of silicone, plastic, metal, rubber, or like materials. The recessed housing structure 24 advantageously provides a sterile enclosure for the cutting tip 20 prior to use and acts as a protective cover for the user and during storage. A resilient spring 26 may also disposed on the support 12 and rest against the housing structure 24. The spring 26 will automatically retract the cutting tip 20 back into the housing structure 24 following intercostal penetration. Additionally, the access punch device 10 may further comprise a moveable handle 28 at the proximal end 14 of the shaft 12, which moves in an axial direction, as depicted by arrow 22. The handle 28 and support 12 will usually be integrally formed, as illustrated in FIG. 1, and will be made of plastic, metal, rubber, or like materials.

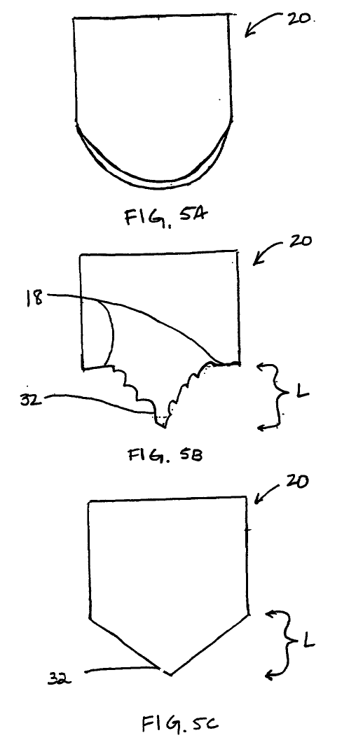

With reference to FIGS. 3 and 4, an alternative cutting tip configuration for access punch 10 comprises two blades 20 and 30. The two blades 20 and 30 will typically be perpendicular to each other to form a cross shape as shown in FIG. 4. The cutting tip 20 will typically have a minimum included diameter D of 5 mm or greater, preferably a minimum diameter D of 1 cm or greater, so that means 18 coupled to cutting tip 20 engage at least one rib.

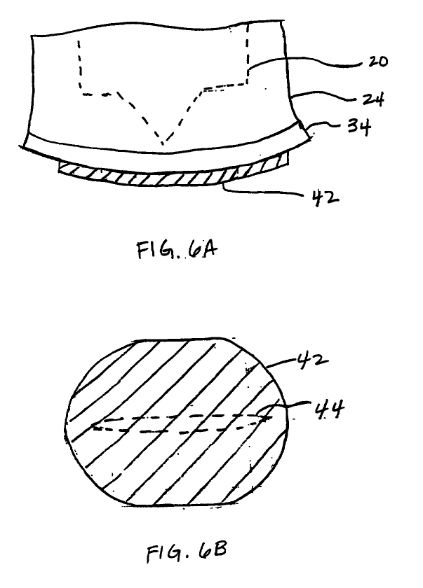

Referring now to FIGS. 5A-5C, cutting tip 20 may comprise a blade having cutting edges that are curved (FIG. 5A), serrated (FIG. 5B), triangular (FIGS. 5B and 5C), or a combination thereof. In particular, the at least one cutting tip 20 will usually form a leading distal tip 32 (i.e. sharp triangular or arrow pointed tip). The triangular distal tip 32 will typically have a maximum length L of 10 mm or less, preferably a maximum length L of 5 mm or less, from the stopping means or shoulder 18 of the cutting tip 20 to further ensure that the cutting tip tip 32 does not accidentally puncture the heart or lungs.

To promote sterility of the presently claimed access punch 10, a bottom side of the recessed housing structure 24 may be enclosed by having a first penetrable cover or membrane 34 disposed over at least a bottom side of the housing structure 24, and a second cover 36 disposed over the first cover 34. The second cover 36 may additionally have a pull-off loop 40 or tab attached to it. It will be appreciated that the covers 34, 36 may be separate components or formed integrally with the housing structure 24. Suitable materials for the membranes include latex, polyethylene, polypropylene, polyester, and the like. The access punch device 10 may further comprise an outer pouch or bag 38 enclosing at least a bottom portion of the recessed housing structure 34, as shown in FIG. 1, or over the entire structure of the device 10, as depicted in FIG. 3. As described above, an important advantage of dual covers 34, 36 and/or an outer pouch 38 is that such membranes eliminate the need for surgical gloves while providing and maintaining a sterile environment before and during use of the access device 10.

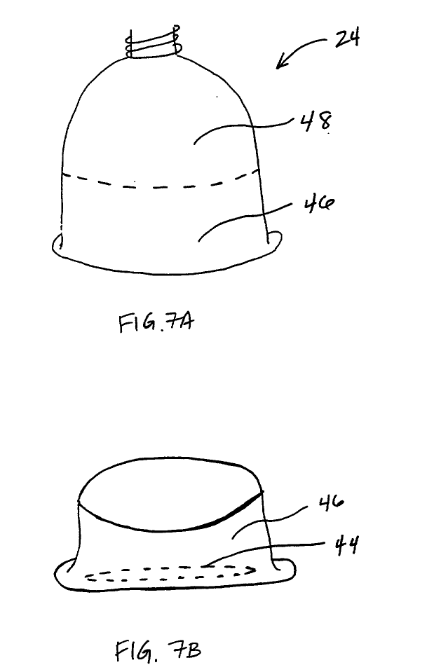

Referring now to FIGS. 6A and 6B, the access punch device 10 is illustrated with a detachable adhesive skin contacting surface 42. The skin contacting surface 42 may be a part of the first cover 34, as illustrated, or a part of the housing structure 24. The adhesive skin contacting surface 42 will typically form a patch around the dissected tissue 44 to maintain near normal inter-thoracic pressure. The adhesive skin contacting surface 42 will be made of suitable materials, such as, hydrogel, adhesive coated paper, rubber, plastic, and the like.

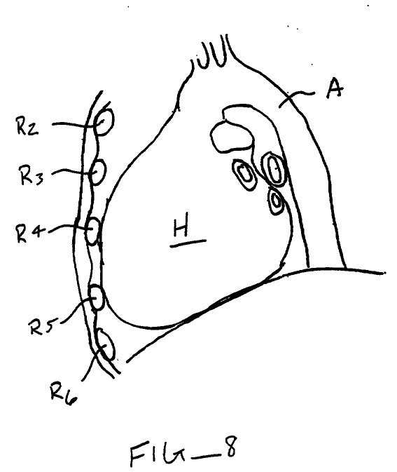

Referring now to FIGS. 7A and 7B, the housing structure 24 of the access punch device 10 may alternatively have a bottom portion 46 which is detachable from a top portion 48 of the housing structure 24 to form an access port 46 over the dissected tissue 44. An access port 46 allows for subsequent entry of devices such as minimally invasive direct cardiac massagers, chest tubes, defibrillator electrodes, and the like.

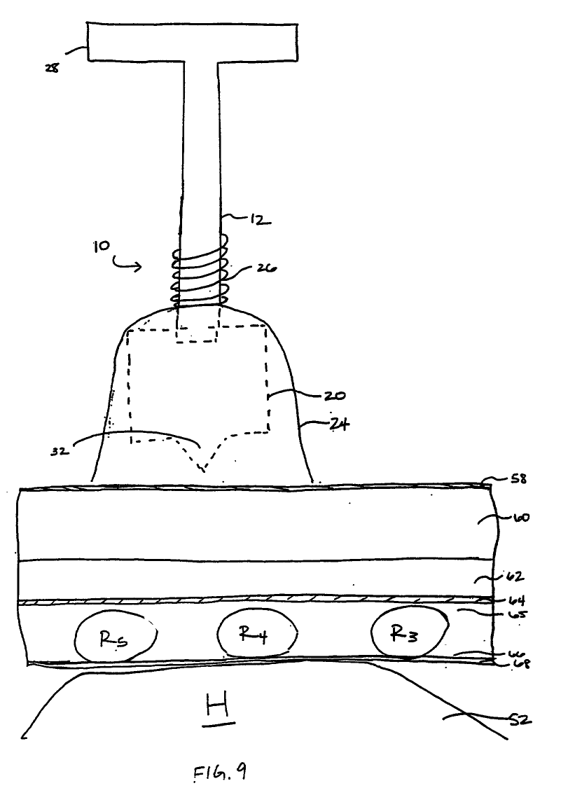

Referring now to FIG. 8, a patient's heart H is shown in a cross-section between ribs Rₙ where n indicates the rib number. The aorta A is also shown extending from the top of the heart.

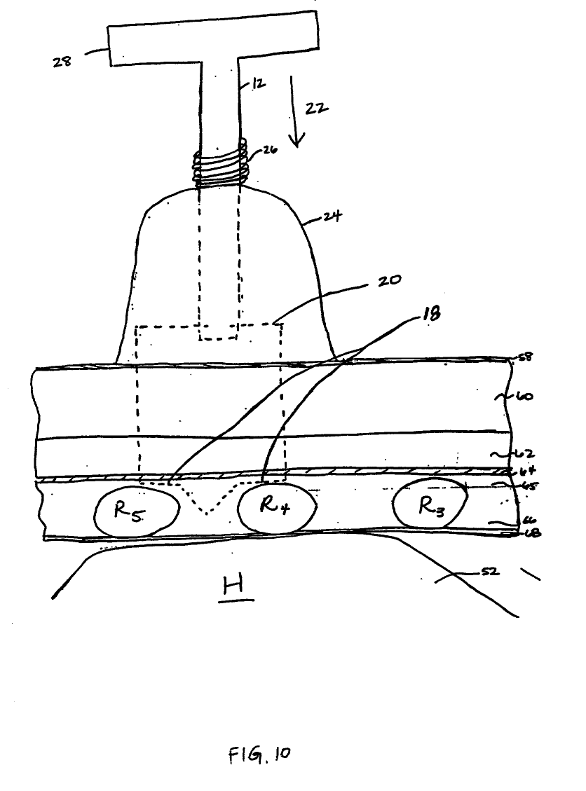

Referring now to FIGS. 9 and 10, a first method for establishing percutaneous intercostal access with the access punch device 10 of FIG. 1 will be described. A cutting tip 20 having a tissue penetrating stop element 18 coupled to the cutting tip 20 is advanced through intercostal tissue. The stop element 18 engages at least one rib after the cutting tip 20 dissects tissue between adjacent ribs but before the cutting tip 20 enters a thoracic cavity 52. Typically, the stop element 18 of the cutting tip 20 will rest against rib R₄ or R₅.

The advancing step is typically carried out by pressing a housing structure 24 against a skin surface 58 and depressing a moveable handle 28 attached to the cutting tip 20 so that the cutting tip 20 cuts through the skin surface 58 to a preselected tissue depth external to the thoracic cavity 52. As illustrated in FIG. 10, a subcutaneous fat layer 60, a muscle layer 62, and a thin facia layer 64 lie respectively beneath the skin surface 58. Cutting tip penetration may be limited to a preselected tissue depth in a range typically from 2 mm to 50 mm, preferably in a range from 2 mm to 15 mm, below an outer rib surface 65. As noted earlier, the exact depth of penetration depends ultimately on the specific patient as there is individual variability in the depth of the thoracic cavity. The preselected tissue depth will usually comprise an inner rib surface 66, as shown, or may alternatively comprise a thoracic pleural lining 68. Typically, the amount of force required to depress the handle 28 in direction 22 will be in a range from 0.5 lbs. to 3 lbs. Preferably, the advanced cutting tip 20 is automatically retracted back into the housing structure 24 via a resilient spring 26 (FIG. 9). The resilient spring 26 will typically require 0.25 lbs. to 1 lbs. of force to be fully compressed.

Preferably, an outer pouch 38 disposed over at least a bottom portion of the housing structure 24 will be removed prior to advancing the cutting tip 20. An outer cover 36 disposed on a bottom side of the housing structure 24 may also be removed via a pull-off loop 40 or tab prior to advancing the cutting tip 20. The cutting tip 20 may also cut through an inner cover 34 enclosing the bottom side of the housing structure 24 prior to cutting through the skin surface 58 to the preselected tissue depth.

Following cutting tip advancement, an adhesive skin contacting surface 42 of the inner cover 34 or the housing structure 24 may be detached on the skin surface 58 so as to form an access patch around the dissected tissue 44. Optionally, a bottom portion 46 of the housing structure 24 may be detached from a top portion 48 of the housing structure 24 so as to form an access port over the dissected tissue 44. Still further optionally, a direct cardiac massage device may be advanced following intercostal dissection.

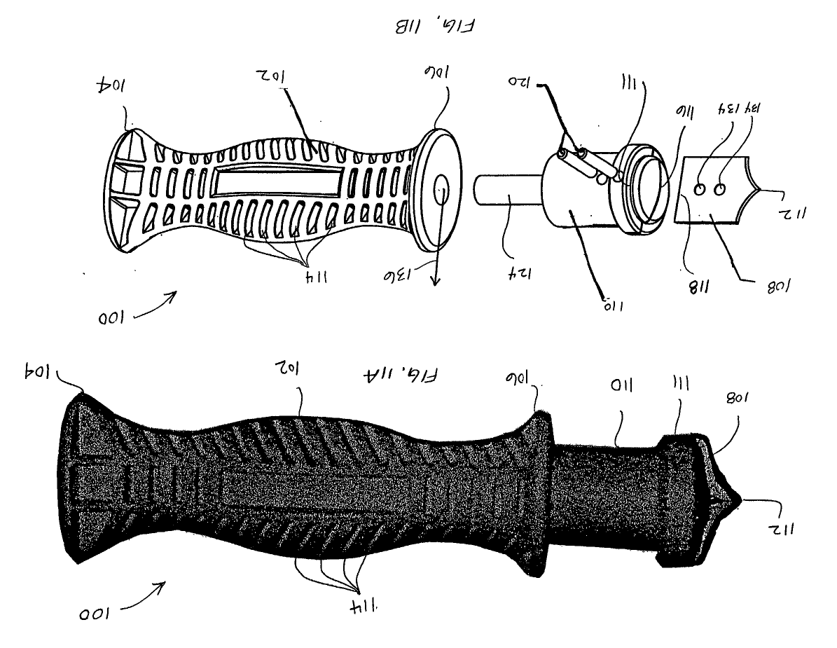

Referring now to FIGS. 11A and 11B, an exemplary access device 100 constructed in accordance with the principles of the present invention will be described. The intercostal access device 100 comprises handle a 102 having a proximal end 104 and a distal end 106, a cutting tip 108 coupled to the distal end 106 of the handle, and rib engaging means coupled to the handle 102 and/or the cutting tip 108. The cutting tip 108, which may comprise a blade, scalpel, or trocar, is adapted to penetrate percutaneously through thoracic tissue between the skin surface and ribs to a thoracic cavity. Means coupled to the handle 102 and/or the cutting tip 108 engage at least one rib and stop advancement of the cutting tip 108 into the thoracic cavity.

In this embodiment, the rib engaging means to stop advancement of the cutting tip 108 into the thoracic cavity takes the form of a cylindrical structure 110 having a rib engaging surface 111 which is formed separately from the cutting tip 108. Such a separate structure 110 mechanically couples the handle 102 to the cutting tip 108 so that the structure 110 will separate or dilate tissue when the cutting tip 108 is advanced and engage an anterior rib surface to halt advancement of the cutting tip 108 at an appropriate point, i.e., as a leading tip 112 of the cutting tip 108 reaches a preselected intercostal depth (typically being in a range from about 2 mm to about 15 mm from an outer rib surface). The cylindrical structure 110 will have a length in the range from 20 mm to 40 mm, a diameter greater than 10 mm, preferably in the range from 20 mm to 40 mm to ensure that the cylindrical structure 110, 111 engages at least one rib, and will be formed from plastic, metal, composite, or like materials.

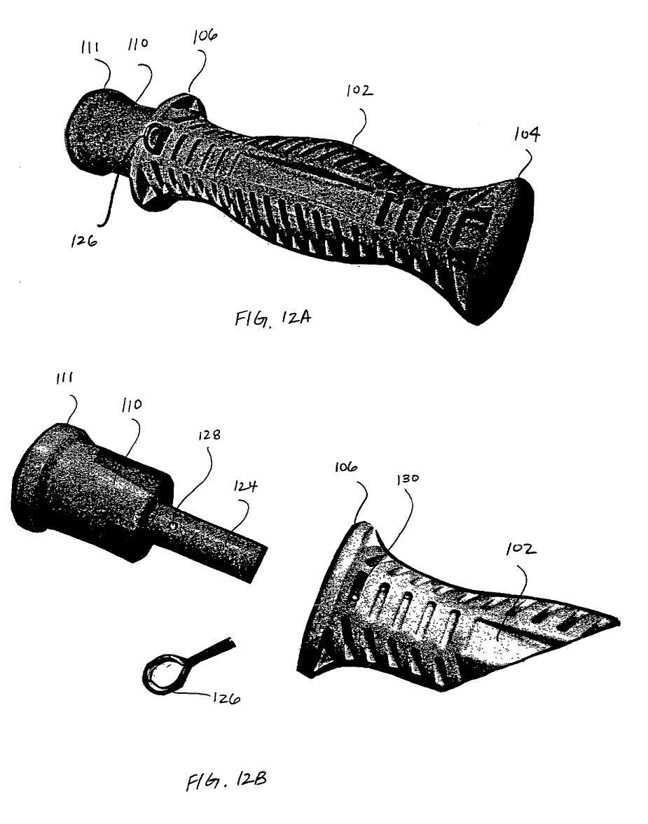

As illustrated in FIG. 11B, a proximal end 124 of the cylindrical structure 110 may be removably attachable to the distal end 106 of the handle 102. It will be appreciated by those of skill in the art that there are numerous ways to removably couple the structure to the handle, such as, a pressure fit (FIG. 11B), latch mechanism, cross pin mechanism, and the like. Preferably, as illustrated in FIGS. 12A and 12B, the cylindrical structure 110 is removably coupled to the handle 102 by a cross pin configuration 126. As shown in FIG. 12B, the cylindrical structure 124 has a first hole 128 and the distal end of the handle 106 has another hole 130 for receiving a handle pin 126 so that the cylindrical structure 124 is secured within the handle 102. The present access device 100 advantageously allows for the cylindrical structure 110 and cutting tip 108 to be separable from the handle 102 after use via removal of the cross pin 126. This in turn allows the cylindrical structure 110 and cutting tip 108 to be easily disposed of in a sharps container, as conventional sharps containers may not allow for disposal of the whole access device unit 100 due to size constraints. This removability feature further allows the handle 102 to be separately disposed of or reused (after undergoing autoclaving or other sterilization procedures) once it has been uncoupled from the cylindrical structure 110.

The handle 102 will usually comprise a shaft or support structure which may have a generally oval shape so as to provide a comfortable and ergonomically correct fit to a user of the device. The handle's oval cross-section has a longitudinal axis 136 which may be alignable with the cutting tip 108 to facilitate orientation of the cutting tip 108. The handle will have a length in the range from 70 mm to 140 mm, a width in the range from 10 mm to 40 mm, and will be formed from plastic, metal, rubber, or like materials. The handle 102 may additionally have ribs 114 or strips on an outer surface thereof to facilitate gripping of the access device 100.

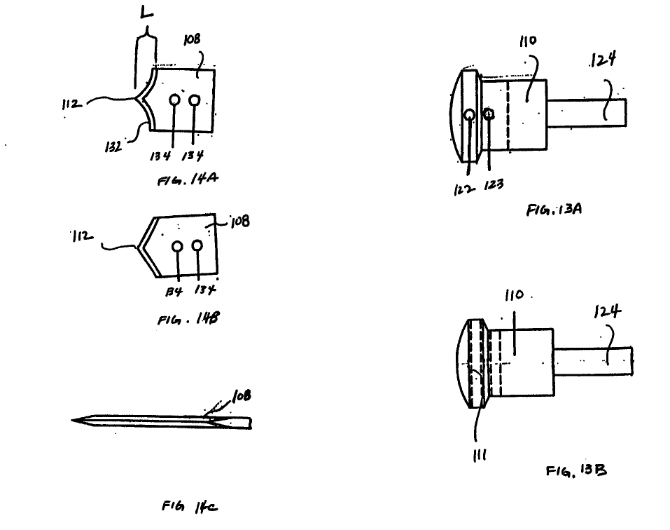

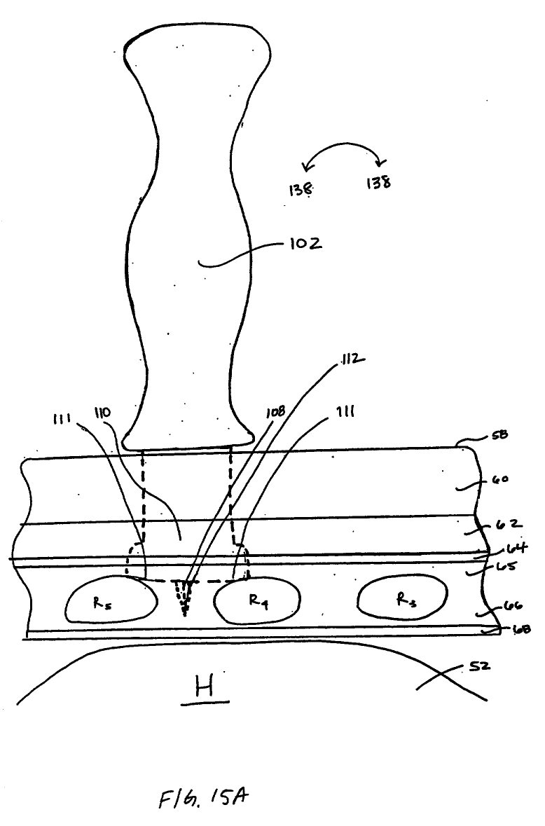

The cylindrical structure 110 may further act as a cutting tip holder by having a slot 116, opening, hole, gap, or slit on a distal end thereof for receiving a proximal end 118 of the cutting tip 108, wherein the cutting tip 118 is secured within the cylindrical structure 110 by a cutting tip dowel 120 or pin or any other mechanical coupling mechanism. The cylindrical structure 110 has a second and third hole 122, 123, as illustrated in FIGS. 13A and 13B, and the cutting tip 108 has another two holes 134, as shown in FIG. 14A, for receiving the cutting tip dowels 120 or pins. In some instances, the cutting tip 108 may be independently separable from the cylindrical structure 110 after use via removal of the cutting tip dowels 120. Hence, the handle 102, the cylindrical structure 110, and the cutting tip 108 may be fixed relative to one another during use, wherein the cutting tip 108 remains in an exposed position. Before and after use, all device components may be uncoupled from one another so that the cutting tip 108, the cylindrical structure 110, and/or the handle 102 may be separately stored or disposed of. In other instances, however, it may be desirable to have the handle 102, cylindrical structure 110, and the cutting tip 108 integrally formed as a single unit (not shown).

Referring now to FIGS. 14A through 14C, the cutting tip 108 will preferably comprise a triangular blade having cutting edges which may be slightly curved (FIG. 14A) or straight (FIG. 14B) and a leading end which is pointed 112. The leading point 112 will typically have a maximum length L of 10 mm or less, preferably a maximum length of 6 mm or less, from a shoulder 132 or lateral extension of the cutting tip 108 to further ensure that the cutting tip 108 does not accidentally puncture the heart or lungs. The cutting tip 108 will have a length in the range from 10 mm to 30 mm, a width that is sufficiently wide to allow for passage of the cylindrical structure 110, preferably a width in the range from 10 mm to 40 mm, and a thickness in the range from 0.1 mm to 1.0 mm.

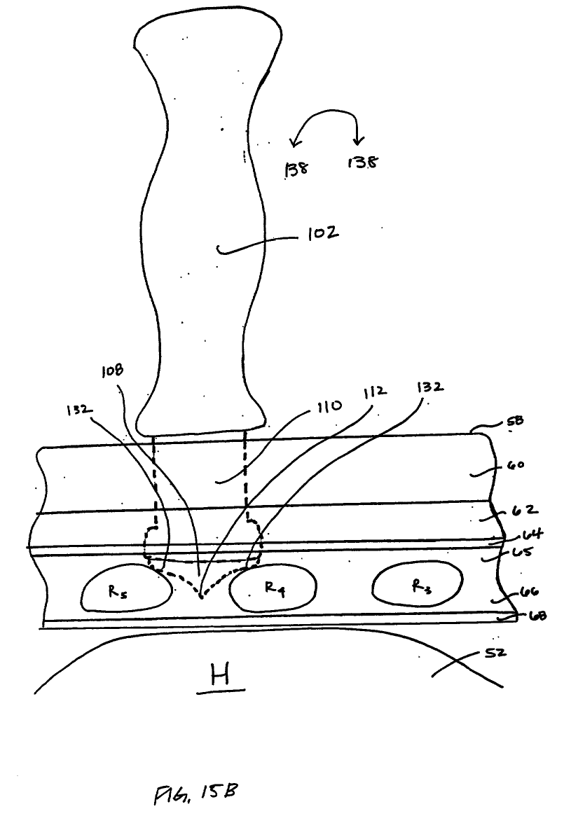

Referring now to FIGS. 15A and 15B, methods for establishing percutaneous intercostal access with the access device of FIG. 11A will be described. The intercostal access device 100 may establish a percutaneous intercostal tract by advancing a cutting tip 108 having a rib engaging stop element coupled to the cutting tip 108 percutaneously through thoracic tissue (i.e. through the skin surface 58 to a preselected intercostal tissue depth external to the thoracic cavity 52). As illustrated in FIG. 15A, the rib engaging stop element comprises a cylindrical structure 110, 111 . The rib engaging surface 111 of the stop element 110 engages at least one rib, typically the third, fourth, or fifth rib, after the cutting tip 108 sharply dissects tissue between adjacent ribs but before the cutting tip 108 enters a thoracic cavity 52. In this embodiment, the cutting tip 108 is aligned with the ribs, R₄ and R₅, prior to cutting tip advancement so that the cutting tip 108 cuts in a plane parallel to the ribs. Such cutting tip orientation may be desirable as an access incision that lies in a plane parallel to the ribs allows for improved suturing and healing of the thoracic tissue post treatment. Cutting tip orientation may further be facilitated by the oval cross-section handle. The advancing step may be carried out by moving the handle 102 in a rocking motion 138 so that the cutting tip 108 effectively cuts through the skin surface 58, subcutaneous fat layer 60, and muscle layer 62 to a preselected tissue depth external to the thoracic cavity 52. The advancing step may further comprise dilating or separating the thoracic tissue with the stop element 110 as the leading tip 112 of the cutting tip 108 is percutaneously advanced.

Referring now to FIG. 15B, the rib engaging stop element may alternatively comprise a lateral extension or shoulder 132 of the cutting tip 108. The lateral extension 132 is configured to penetrate the thoracic tissue and to engage an anterior surface of the rib before the leading tip 112 of the cutting tip 108 penetrates too far. Cutting tip penetration may be limited to a preselected intercostal tissue depth in a range typically from 2 mm to 50 mm, preferably in a range from 2 mm to 15 mm, below the outer rib surface 65. The preselected tissue depth will usually comprise tissue near an inner rib surface 66, or may alternatively comprise a thoracic pleural lining 68. Still further the preselected tissue depth may be within a range from 3 mm to 5 mm from the thoracic cavity 52.

Following establishment of the intercostal access tract, the cutting tip 108 may be uncoupled from the handle 102 by removing the cross pin 126 from the handle 102. The cutting tip 108 and cylindrical structure 110 can then be separately disposed of in a conventional sharps container and the handle 102 reused. Optionally, in some instances the cutting tip 108 may be further separable from the cylindrical structure 110 by removing the cutting tip dowels 120 from the structure 110. A direct cardiac massage device, chest tube, or other percutaneous devices may then be advanced following intercostal advancement.

Although certain preferred embodiments and methods have been disclosed herein, it will be apparent from the foregoing disclosure to those skilled in the art that variations and modifications of such embodiments and methods may be made without departing from the true spirit and scope of the invention. For example, the access device of FIG. 11A may further incorporate a flexible housing structure for housing the cutting tip. Therefore, the above description should not be taken as limiting the scope of the invention which is defined by the appended claims.

What is claimed is:

1. An intercostal access device comprising:
a handle having a proximal end and a distal end; and
a cutting tip coupled to the distal end of the handle, said cutting tip being adapted to penetrate percutaneously through thoracic tissue between a skin surface and ribs to a thoracic cavity; and
means coupled to the handle or cutting tip for engaging at least one rib and stopping advancement of the cutting tip into the thoracic cavity.

2. An access device as in claim 1, wherein the rib engaging means comprises a separate cylindrical structure which is configured to separate tissue when the cutting tip is advanced and to engage an anterior surface of a rib as a leading tip of the cutting tip reaches a preselected intercostal depth.

3. An access device as in claim 2, wherein the cylindrical structure couples the handle to the cutting tip.

4. An access device as in claim 2, wherein a proximal end of the cylindrical structure is removably attachable to the distal end of the handle.

5. An access device as in claim 4, wherein the cylindrical structure has a first hole for receiving a handle pin so that the cylindrical structure is secured within the handle.

6. An access device as in claim 4, wherein the cylindrical structure and cutting tip are separable from the handle.

7. An access device as in claim 2, wherein a distal end of the cylindrical structure has a slot to receive the cutting tip.

8. An access device as in claim 7, wherein the cylindrical structure has a second and third hole for receiving a cutting tip pin or dowel so that the cutting tip is secured within the cylindrical structure.

9. An access device as in claim 7, wherein the cutting tip is independently separable from the cylindrical structure.

10. An access device as in claim 2, wherein the cylindrical structure has a diameter greater than 10 mm.

11. An access device as in claim 1, wherein the rib engaging means comprises a lateral extension of the cutting tip which is configured to penetrate tissue and to engage an anterior surface of a rib before a leading tip of the cutting tip penetrates too far.

12. An access device as in claim 1, wherein the handle has a generally oval shape.

13. An access device as in claim 1, wherein the handle has a longitudinal axis which is alignable with the cutting tip to facilitate orientation of the cutting tip.

14. An access device as in claim 1, wherein the handle has ribs or strips on an outer surface thereof.

15. An access device as in claim 1, wherein the handle has at least one hole for receiving a pin.

16. An access device as in claim 1, wherein the handle is reusable.

17. An access device as in claim 1, wherein the cutting tip is a triangular blade.

18. An access device as in claim 1, wherein the cutting tip has a leading end which is pointed.

19. An access device as in claim 18, wherein the leading point has a length less than 6 mm.

20. An access device as in claim 1, wherein the cutting tip has a width greater than 10 mm.

21. An access device as in claim 1, wherein the cutting tip is alignable parallel to the ribs.

22. An access device as in claim 1, wherein the cutting tip has at least one hole for receiving a pin or dowel.

23. An access device as in claim 1, wherein the cutting tip is disposable.

24. An intercostal access device comprising:
a handle having a proximal end and a distal end;
a blade holder removably coupled to the distal end of the handle; and
a blade coupled to a distal end of the holder, wherein the blade has a leading tip sized to advance through intercostal tissue between adjacent ribs and the blade holder is positioned to engage at least one rib and stop advancement of the leading tip at a preselected intercostal depth as the blade is percutaneously advanced.

25. An intercostal access device comprising:
a handle having a proximal end and a distal end;
a blade holder removably coupled to the distal end of the handle; and
a blade removably coupled to a distal end of the holder, wherein the blade has a leading tip and at least one shoulder, wherein the leading tip is sized to advance between adjacent ribs and the shoulder is positioned to engage at least one rib and stop advancement of the leading tip prior to entry of the leading tip into a thoracic cavity as the blade is percutaneously advanced toward a heart.

26. A method for percutaneous intercostal access, said method comprising:
advancing a cutting tip having a rib engaging stop element coupled to the cutting tip percutaneously through thoracic tissue, wherein the stop element engages at least one rib after the cutting tip dissects tissue between adjacent ribs.

27. A method as in claim 26, wherein the stop element engages at least one rib before the cutting tip enters a thoracic cavity.

28. A method as in claim 26, wherein the advancing step is carried out by moving a handle coupled to the cutting tip by a cutting tip holder in a rocking motion so that the cutting tip cuts through the thoracic tissue to a preselected intercostal depth.

29. A method as in claim 28, wherein the advancing step further comprises dilating the tissue with the cutting tip holder as the cutting tip is percutaneously advanced.

30. A method as in claim 28, further comprising uncoupling the cutting tip from the handle following intercostal advancement.

31. A method as in claim 30, wherein the uncoupling comprises removing a pin from the handle.

32. A method as in claim 31, further comprising disposing the cutting tip and holder in a sharps container.

33. A method as in claim 31, further comprising reusing the handle.

34. A method as in claim 28, wherein the preselected intercostal depth comprises tissue near an inner rib surface.

35. A method as in claim 28, wherein the preselected intercostal depth comprises a thoracic parietal pleural lining.

36. A method as in claim 28, wherein the preselected intercostal depth is in a range from 2 mm to 15 mm from an outer rib surface.

37. A method as in claim 28, wherein the preselected intercostal depth is in a range from 3 mm to 5 mm from a thoracic cavity.

38. A method as in claim 26, further comprising aligning the cutting tip with the ribs prior to cutting tip advancement so that the cutting tip cuts in a plane parallel to the ribs.

39. A method as in claim 26, further comprising advancing a chest tube following intercostal advancement.

40. A method as in claim 26, further comprising advancing a direct cardiac massage device following intercostal advancement.
